(19) United States
(12) Patent Application Publication
(10) Pub. No.: US 2021/0236658 A1
(43) Pub. Date: Aug. 5, 2021

(54) VISUALIZING AGENT FOR VISUALIZING HYALURONAN

(71) Applicant: Fenelon Holland Holding B.V., Rotterdam (NL)

(72) Inventor: Gerrit Reinold Jacob MELLES, Rotterdam (NL)

(21) Appl. No.: 17/051,985

(22) PCT Filed: May 3, 2019
(86) PCT No.: PCT/NL2019/050265

§ 371 Date: Oct. 30, 2020

(30) Foreign Application Priority Data
May 4, 2018 (EP) 18170794.4

(51) Int. Cl. A61K 49/00; A61K 6/25; C08G 73/02

(52) U.S. Cl. CPC A61K 49/0054; C08G 73/0233; A61K 6/25; A61K 49/006

(57) ABSTRACT

The invention is directed to a visualizing agent comprising a polyoxazoline molecule and one or more labeling compounds. The visualizing agent can be an ocular staining composition that can be used to stain the vitreous body.

The invention is directed to a molecular complex for visualizing hyaluronan tissue (in particular the vitreous body in the eye), a composition for visualizing hyaluronan tissue, a method for preparing the complex and the composition, and the use of the complex and composition in surgery, diagnostics, therapeutics and cosmetics.

Hyaluronan, also called hyaluronic acid, is a polysaccharide consisting of a repeating disaccharide unit. The disaccharide unit in hyaluronan is composed of D-glucuronic acid and N-acetyl-D-glucosamine linked via glycosidic bonds. Hyaluronan belongs to the group of mucosaccharides. It is highly polar in an aqueous environment due to the presence of negatively charged groups.

Hyaluronan is present in certain tissue of the human and animal body. The vitreous body located in the posterior segment of the eye contains large amounts of hyaluronan. Hyaluronan is also an important component of articular cartilage (and hyaline cartilage in particular), wherein it is present at the outer surface of chondrocyte cells. Hyaluronan is also present in certain tissue in the mouth. Most periodontal tissues comprises hyaluronan, and especially the gingiva (the gums) consists of large amounts of hyaluronan. Further, hyaluronan is present in significant amounts in the skin, hair follicles and connective tissues.

Visualizing agents, such as dyes and contrast agents, are used to visualize certain tissues in order to distinguish them from other tissues. There is a need in the art to selectively apply visualizing agents to hyaluronan tissue. For example, if one would be able to stain the gingiva with a dye, while the surrounding tissues remain unstained, this may facilitate dental surgery. Similarly, it would be helpful to selectively apply a visualizing agent such as a contrast agent to hyaline cartilage to diagnose and/or monitor certain diseases. Further, it may aid various surgical procedures when hyaline ‘injectables’ can be visualized during or after the procedure, to locate their presence (in case hyaline injectables are used as a therapeutic tool) or confirm their absence (in case hyaline injectables have to be removed again from the tissues operated on).

In particular, there is a strong need in eye surgery to be able to visualize hyaluronan tissue. It is common practice in eye surgery to apply a dye to specific ocular structures to help the surgeon visualize and distinguish the different ocular tissues. For example, it is common practice to stain the anterior lens capsule (located in the anterior segment) with a dye in order to facilitate performing capsulorhexis. This procedure is for example described in WO 99/58160. The method described in WO 99/58160 uses a vital dye that is capable of staining tissue without diffusing through said tissue. Furthermore, it is known to stain a retinal membrane (located in the posterior segment) to facilitate vitreo-retinal surgery. Examples of such membranes are the inner limiting membrane and the epiretinal membrane. Examples of vitreo-retinal surgery that may be facilitated are retinal detachment surgery, macula pucker removal and macular hole surgery. This type of staining is described in WO 99/58159.

A shortcoming of the procedures of WO 99/58159 and WO 99/58160 is that the staining method and dye compositions described therein are not capable of staining the vitreous body.

The vitreous body is a tissue that consists of a clear, transparent, semi-solid gel, which is located between the crystalline lens and the retina in the posterior segment of the eye. The vitreous body serves as a space maintainer in the posterior segment of the eye. It is mainly composed of hyaluronan with only very small amounts of fibrous structures, such as collagen and vitrosin, glucose and trace elements. Because its refractive index nearly equals that of the crystalline lens and of aqueous as well as balanced salt solutions during surgery, it is virtually impossible to visualize the vitreous body or elements thereof and to discriminate it from the surrounding liquid elements even at high magnification through a surgical microscope. During the first decades of life, the vitreous body is attached to the retina, which may further hinder differentiation of these anatomical structures.

Accordingly, there is a need for a dye composition that is capable of visualizing the vitreous body.

The present invention seeks to overcome the above described problems associated with insufficient visibility of the vitreous body or parts thereof during surgery.

An object of the invention is to provide a compound or composition capable of visualizing hyaluronan containing tissue.

A further object of the invention is to provide a compound or composition capable of improving visualization of separate ocular tissues during eye surgery. In particular, it is an object of the invention to make it possible to visualize the vitreous body, as well as to enable the surgeon to distinguish the vitreous body from the surrounding ocular structures of secondarily formed fibrotic tissue during surgery, and to provide sufficient contrast to facilitate selective removal of the vitreous element.

A further object of the invention is to provide a compound or composition capable of staining the vitreous body.

One of these objects was met by providing a visualizing agent comprising a polyoxazoline molecule and one or more labeling compounds. Accordingly, in a first aspect, the invention is directed to a visualizing agent comprising one or more labeling compounds that are molecularly bound to a polyoxazoline molecule having a repeat unit according to the formula

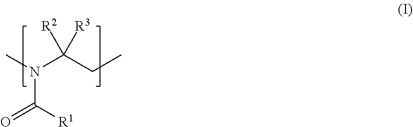

wherein R¹ is an alkyl group or a phenyl group, wherein said alkyl or phenyl is optionally substituted with one or more substituents selected from the group consisting of halogen, amino (—NH₂), nitro (—NO₂), carboxyl (—COOH), alkoxy (—OR), sulfonate (—SO₃⁻), hydroxyl (—OH) and sulfhydryl (—SH); and wherein said phenyl may additionally or alternatively also be optionally substituted with one or more alkyl groups; R² and R³ are each individually selected from the group consisting of hydrogen, alkyl, phenyl, halogen, amino (—NH₂), nitro (—NO₂), carboxyl (—COOH), alkoxy (—O—R), sulfonate (—SO₃⁻), hydroxyl (—OH), imino (═N—H) and sulfhydryl (—SH).

The inventors found that the visualizing agent according to the invention is capable of staining the vitreous body without affecting or staining the surrounding ocular tissue or structures. This allows for improved contrast between the vitreous body and the surrounding ocular tissues such as the retina. To the inventors' knowledge, this is the first known agent capable of effectively staining the vitreous body.

However, the invention is not limited to staining the vitreous body. The inventors found that the polyoxazoline molecule present in the visualizing agent is capable of staining the vitreous body due to the presence of hyaluronan in this tissue. Accordingly, the complex of the invention is expected to also be capable of visualizing hyaluronan in general. Thus, the invention not only opens up visualizing opportunities in eye surgery, but also in other areas wherein visualization of tissues is helpful. The visualizing agent can be used to visualize hyaluronan containing tissue, as well as synthetic hyaluronan. For example, the visualizing agent can be used to visualize tissue in surgery or diagnostics. The visualizing agent can also be used in diagnostic compositions (e.g. in staining compositions or in contrast medium), pharmaceutical compositions (such as in injectionables) and in cosmetic compositions (e.g. for injection in the skin) The visualizing agent may for example be used as a staining compound in dental surgery or as a contrast medium in radiography, X-ray, positron-emission tomography (PET) or magnetic resonance imaging (MRI). Furthermore, the complex formed between polyoxazoline and hyaluronan can be incorporated in pharmaceutical compositions comprising unbound hyaluronan, which composition are suitable for injection into the body.

Without wishing to be bound by any theory, it is believed that the polyoxazoline present in the visualizing agent is capable of binding to the hyaluronan via a non-covalent bond. The bond is expected to be caused by Van der Waals forces, in particular by hydrogen bonds and hydrophobic interactions, between polyoxazoline and hyaluronan. Thus, the polyoxazoline can function as a carrier for the labeling compound. This provides the visualizing agent to visualize hyaluronan tissue. The bond formed between the visualizing agent and hyaluronan tissue is reversible. To the inventors' knowledge, this is the first time that such interaction between polyoxazoline and hyaluronan is described in the art.

The term “visualizing agent” as used herein refers to an agent suitable for visualizing hyaluronan tissue. In particular, the agent is capable of visualizing hyaluronan tissue when brought into contact with the tissue. The type of visualization achieved by the visualizing agent depends on the type of labeling compound present in the agent. The visualizing agent may be molecular complex (wherein the one or more labeling compounds are non-covalently bound to the polyoxazoline) or a compound (wherein the one or more labeling compounds are covalently bound to the polyoxazoline).

The term “hyaluronan tissue” as used herein refers to hyaluronan containing tissue. The term encompasses tissues containing hyaluronan as one of its components, as well as tissues covered with hyaluronan. The tissue may be solid, a gel (e.g. the vitreous), or a fluid (e.g. a body fluid). The tissue can be a connective tissue. Examples of hyaluronan tissue are the vitreous body, the gingiva (gums) and hyaline cartilage (e.g. articular cartilage). The tissue may be animal tissue, in particular mammalian tissue, preferably human tissue. Hyaluronan is also called hyaluronic acid in literature. The two names refer to the same compound and may be used interchangeably.

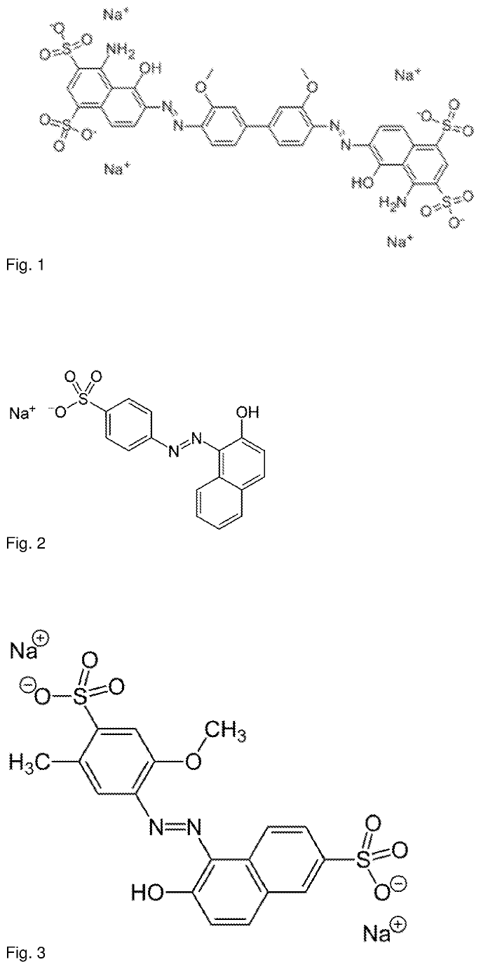

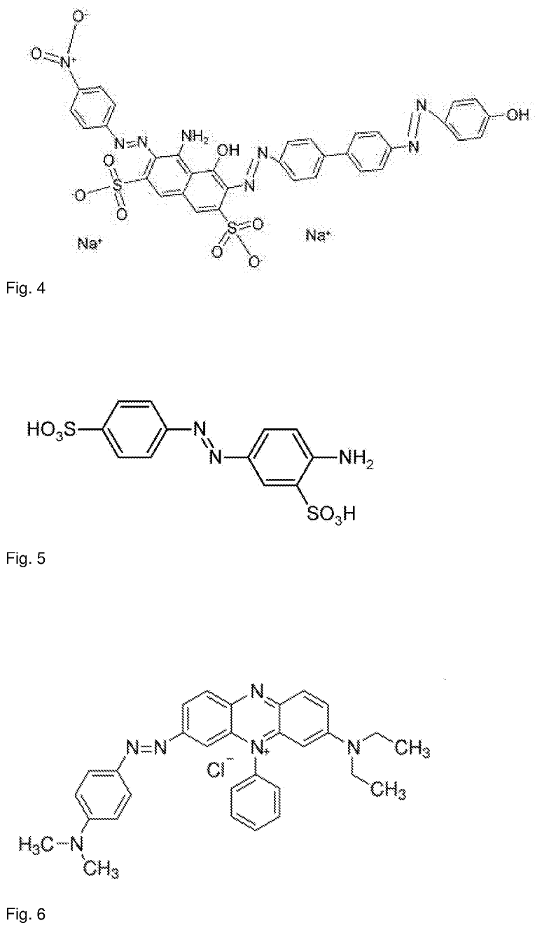

FIGS. 1-6 show molecular structures of six suitable labeling compounds. The chemical structures depicted are Chicago Sky Blue 6B (FIG. 1), 2-naphtol orange (FIG. 2), Allura Red AC (FIG. 3), Diamine Green B (FIG. 4), Fast Yellow AB (FIG. 5) and Janus Green B (FIG. 6).

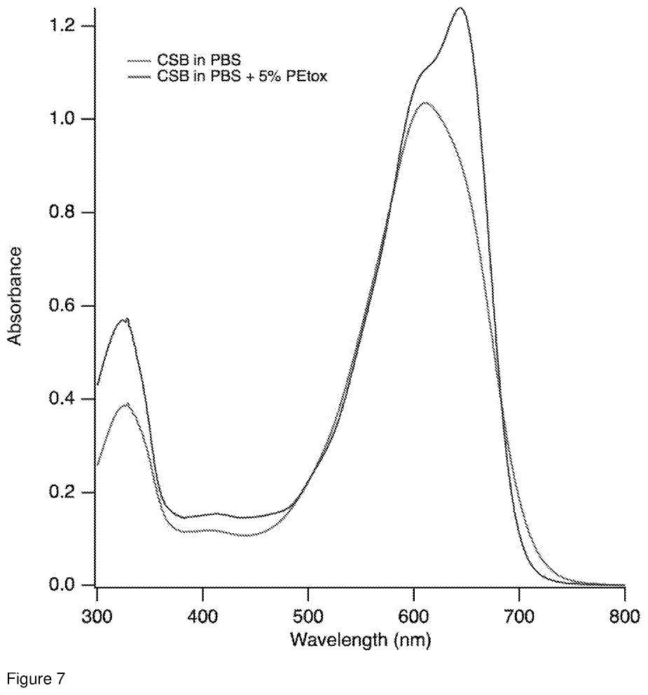

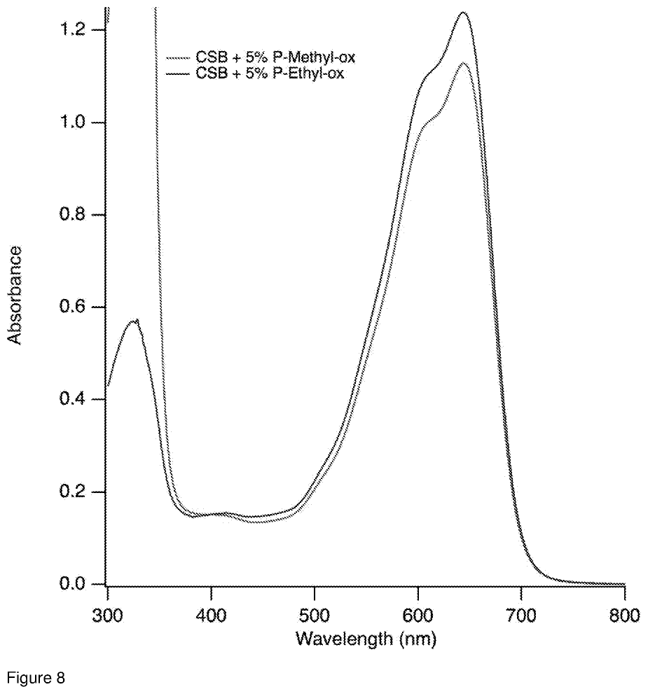

FIGS. 7 and 8 show the results absorbance tests conducted with Chicago Sky Blue and polyoxazoline.

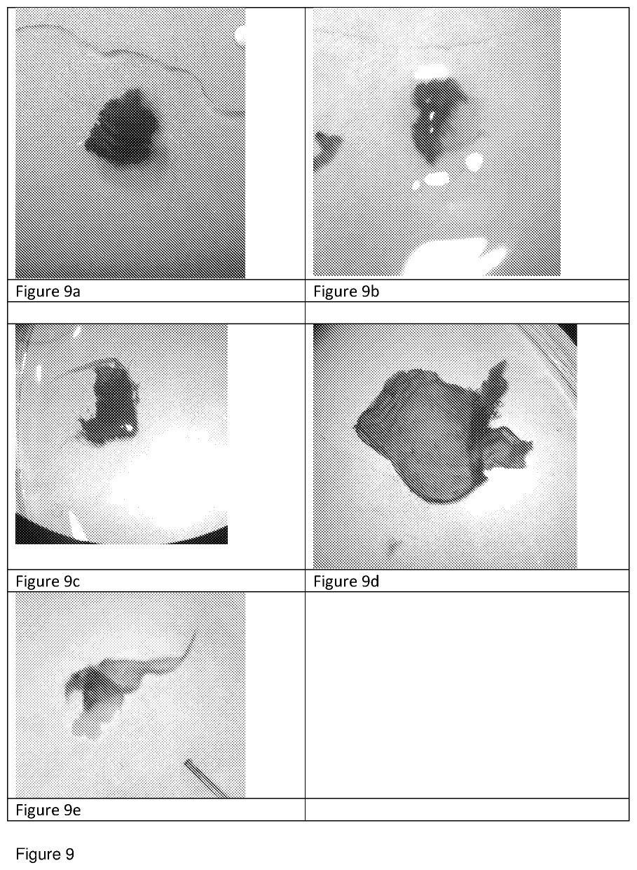

FIG. 9 shows the results of staining experiments of the vitreous body using different azo compounds.

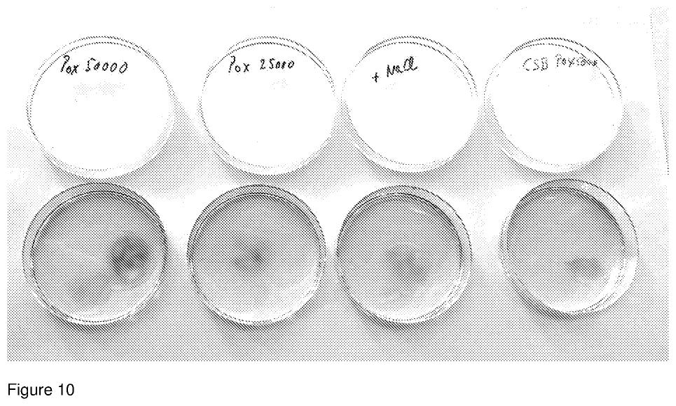

FIG. 10 shows the results of staining experiments of the vitreous body using polyoxazoline of different lengths.

The visualizing agent comprises one or more labeling compounds and the polyoxazoline having a repeat unit according to formula (I). The labeling compounds are for visualizing the hyaluronan tissue. The labeling compounds are molecularly bound to the polyoxazoline. The one or more labeling compounds may be non-covalently bound to the polyoxazoline. For example, the one or more labeling compounds and the polyoxazoline may form a molecular complex, e.g. due to Van der Waals forces, in particular due to hydrogen bonds and hydrophobic interactions. Alternatively, it is also possible that the one or more labeling compounds are covalently bound to the polyoxazoline. It is expected that for both of these cases, the polyoxazoline part of the visualizing agent will be capable of binding to hyaluronan tissue.

The visualizing agent of the invention typically comprises multiple labeling compounds per polyoxazoline. Each labeling compound in the visualizing agent may be covalently or non-covalently bound to the polyoxazoline. The visualizing agent may comprise multiple labeling compounds. In this case, the visualizing agent may comprise 0.01-1, preferably 0.1-0.75, more preferably 0.25-0.5 labeling compounds per polyoxazoline monomer. The number of polyoxazoline monomers in the polyoxazoline may be represented by parameter n. The visualizing agent may comprise at least 2, preferably at least 5, more preferably at least 10 labeling compounds per polyoxazoline. For example, in case of a polyoxazoline having a molecular weight of about 5,000 (i.e. n is about 50), typically about 20 azo dye molecules are bound per polyoxazoline molecule. The visualizing agent may comprise a single or multiple polyoxazoline molecules.

The visualizing agent does not require other components than the one or more labeling compounds and the polyoxazoline. Accordingly, the visualizing agent in its simplest form consists of the one or more labeling compounds and the polyoxazoline.

According to formula (I), R¹ may be an alkyl or phenyl, wherein said alkyl or phenyl is optionally substituted with one or more substituents selected from the group consisting of fluoro, chloro, bromo, amino (—NH₂), nitro (—NO₂), carboxyl (—COOH), methoxy (—O—CH₃), ethoxy (—O—CH₂—CH₃), sulfonate (—SO₃⁻), hydroxyl (—OH) and sulfhydryl (—SH). Additionally or alternatively, phenyl may also be optionally substituted with one or more methyl or ethyl groups.

Preferably, R¹ is alkyl, which may optionally be substituted as described above. R¹ may have 1 to 8 carbon atoms, and preferably has 1 to 5 carbon atoms. More preferably, the alkyl group is selected from methyl, ethyl, n-propyl and isopropyl. Most preferably, R¹ is methyl or ethyl. Polyoxazolines with such a small alkyl groups have a good solubility in water.

According to formula (I), R² and R³ are each individually selected from the group consisting of hydrogen, alkyl, phenyl halogen, amino (—NH₂), nitro (—NO₂), carboxyl (—COOH), alkoxy (—O—R), sulfonate (—SO₃⁻), hydroxyl —OH), imino (═N—H) and sulfhydryl (—SH). Preferably, at least one of R² and R³ is hydrogen. In case R² or R³ is an alkyl group, the alkyl group is defined in the same way as R¹ (see definition in the previous paragraph). In case R² and/or R³ is an alkoxy group of formula —OR, the R group in this formula is also defined in the same way as R¹. In case R² and/or R³ is halogen, the halogen may be F, Cl, Br, preferably Cl. In case of imino, R² and R³ taken together represent the imino group.

Preferably, R² and R³ are each individually selected from the group consisting of hydrogen, methyl and ethyl. Preferably, at least one of R² and R³ is hydrogen. Even more preferably, R² and R³ are both hydrogen.

The polyoxazoline is preferably a poly(2-alkyl)(2-oxazoline). In this case, R² and R³ in formula (I) are both hydrogen. Excellent visualizing results were achieved using this type of polyoxazoline, as also illustrated in the Examples. Preferably, the polyoxazoline is selected from the group consisting of poly(2-methyl-2-oxazoline), poly(2-ethyl-2-oxazoline), poly(2-n-propyl-2-oxazoline) and poly(2-isopropyl-2-oxazoline). Most preferably, the polyoxazoline is poly(2-ethyl-2-oxazoline).

The polymer may be of a certain length, as represented by the molecular weight or by parameter n. Parameter n represents the number of oxazoline moieties in the polymer. The polyoxazoline can thus be represented by formula (Ib):

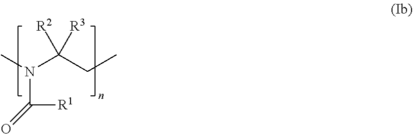

R¹, R² and R³ are as defined above, while n is an integer of typically at least 5. The length of the polymer is mainly determined by the interaction with hyaluronan. A polyoxazoline of too high length may no longer properly bind hyaluronan. Further, a very small polyoxazoline molecule has the disadvantage of being capable of binding relatively few labeling compounds. Accordingly, parameter n is typically in the range of 10 to 5,000, preferably 20 to 1,000, for example 20-500.

The polyoxazoline preferably has a molecular weight (Mw) of 200 to 500,000 g/mol, preferably 500 to 100,000 g/mol, for example 1,000 to 50,000 g/mol.

The labeling compound provides the visualizing agent with the functionality to be visualized. Polyoxazoline itself is not detectable by distinctive color, X-ray or infrared. The labeling compound may be selected from the group consisting of a dye, a radiocontrast agent, an MRI contrast agent, a fluorescent compound, an isotope labeled compound and a cosmetic coloring agent. A dye can be used to visualize a tissue directly by visual inspection. This type of visualization is herein also referred to as staining. A radiocontrast agent can be used to visualize a tissue when subjecting the tissue (and the agent bound thereto) to X-rays. A fluorescent compound can be used to visualize a tissue when subjecting the tissue (and the agent bound thereto) to ultraviolet light. An MRI contrast agent can be used to visualize a tissue when subjecting the tissue (and the agent bound thereto) to magnetic resonance. An isotope labeled compound can be used to visualize a tissue when subjecting the tissue (and the agent bound thereto) to infrared light or nuclear magnetic resonance or by observing the emission of positrons.

Preferably, the labeling compound is an azo compound. Preferably, the azo compound has a moiety according to the formula

[symbol]

Ar₁—N═N—Ar₂ [symbol] (II)

wherein Ar₁ and Ar₂ are the same aromatic ring or different aromatic rings. The aromatic ring can be a monocylic ring (e.g. benzene), a heterocyclic ring (e.g. naphtalene) or a multicyclic ring (e.g. phenazine). In case of a monocyclic ring, the ring is preferably a 5- or 6-membered aromatic ring. In case of a heterocyclic ring, the ring preferably consists of two fused 6-membered rings or a 5 membered ring fused with a 6-membered ring. The aromatic ring may be a homocyclic or a heterocyclic ring. In case of a heterocyclic ring, one or more of the ring members are preferably an element selected from nitrogen (N), oxygen (O) and sulfur (S). The aromatic ring may be substituted with one or more groups other than hydrogen, which one or more groups are preferably each individually selected from methyl, ethyl, phenyl, methoxy, nitro, amino, dimethylamino, diethylamino, hydroxyl, and sulfonate. Such an aromatic ring may be referred to as a substituted aromatic ring.

Ar₁ is preferably a substituted phenyl moiety, a substituted naphtyl moiety, a substituted pyrazole moiety, a substituted benzothiazole or a substituted phenazine moiety. Similarly, Ar₂ is preferably a substituted phenyl moiety, a substituted naphtyl moiety, a substituted pyrazole moiety a substituted benzothiazole or a substituted phenazine moiety. Ar₁ and Ar₂ are preferably each individually selected from a phenyl moiety, a naphtyl moiety, a pyrazole moiety, a benzothiazole moiety and a phenazine moiety, wherein said moieties may optionally be substituted with one or more groups selected from methyl, ethyl, phenyl, methoxy, nitro, amino, dimethylamino, diethylamino, hydroxyl, and sulfonate. The inventors found that such an azo compound was capable of forming a very stable complex with the polyoxazoline. It is expected that this bond is a non-covalent bond, wherein the azo functional group may interact with the amide group in the polyoxazoline. Further, the aryl groups may interact with the hydrophobic chain and groups of the polyoxazoline.

Preferably, Ar₁ and Ar₂ are each individually selected from a phenyl moiety and a naphtyl moiety, wherein said phenyl moiety and napthyl moiety may optionally be substituted with one or more groups selected from methyl, ethyl, methoxy, nitro, amino, hydroxyl, and sulfonate. More preferably, at least one of Ar₁ and Ar₂ is a phenyl moiety optionally substituted with one or more groups selected from methyl, ethyl, methoxy, amino, hydroxyl, and sulfonate.

Examples of compounds having a moiety according to formula (II) are Chicago Sky blue 6B; 2-naphtol orange; Allura Red AC; Diamine Green B; Fast Yellow AB; Janus Green B; Naphtol Blue Black, Tartrazine, Scarlet Red, Thiazole Yellow G and Sudan Black B. Structures of some of these compounds are found in FIGS. 1-6.

The labeling compound is preferably an azo compound having the formula

Ar₃—N═N—Ar₄ (III).

wherein Ar₃ and Ar₄ are the same or different (hetero)aryl groups. The term (hetero)aryl group refers to the group consisting of aryl groups and heteroaryl groups. The (hetero)aryl can be a monocylic group (e.g. benzene), a heterocyclic group (e.g. naphtalene) or a multicyclic group (e.g. phenazine) In case of a monocyclic (hetero)aryl group, the group is preferably a 5- or 6-membered aryl group. In case of a heterocyclic (hetero)aryl group, the group preferably consists of two fused 6-membered rings or a 5 membered ring fused with a 6-membered ring. In case of a heteroaryl group, one or more of the ring members in the group are preferably an element selected from nitrogen (N), oxygen (O) and sulfur (S). The (hetero)aryl group may be substituted with one or more groups other than hydrogen, which one or more groups are preferably each individually selected from methyl, ethyl, phenyl, methoxy, nitro, amino, dimethylamino, diethylamino, hydroxyl, sulfonate, sulfhydryl and —N═N—Ar₅. The group —N═N—Ar₅ is an azo group, wherein Ar₅ is a (hetero)aryl group, preferably selected from a phenyl and naphtyl, wherein said (hetero)aryl group may optionally be substituted with one or more groups selected from methyl, ethyl, methoxy, nitro, amino, hydroxyl, carboxyl, sulfonate and sulfhydryl.

Ar₃ and Ar₄ are preferably each individually selected from phenyl, biphenyl, naphtyl, pyrazole, benzothiazole and phenazine, wherein said phenyl, biphenyl, naphtyl, pyrazole, benzothiazole and phenazine may optionally be substituted with one or more groups selected from methyl, ethyl, phenyl, methoxy, nitro, amino, dimethylamino, diethylamino, hydroxyl, sulfonate, sulfhydryl and —N═N—Ar₅.

Examples of compounds of formula (III) are Chicago Sky blue 6B; 2-naphtol orange; Allura Red AC; Diamine Green B; Fast Yellow AB; and Janus Green B. These dyes are also likely candidates for use in ocular staining compositions, because they provide a clearly visible staining at very low amounts. Also, their clinical use for over 20 years has shown that they have an advantageous toxicity profile. Most preferably, the labeling compound is Chicago Sky Blue 6B.

In a preferred embodiment, Ar₁ and Ar₂ are each individually selected from phenyl and naphtyl, wherein said phenyl and napthyl may optionally be substituted with one or more groups selected from methyl, ethyl, methoxy, amino, hydroxyl, and sulfonate. More preferably, at least one of Ar₃ and Ar₄ is phenyl, which phenyl is optionally substituted with one or more groups selected from methyl, ethyl, methoxy, amino, hydroxyl, and sulfonate. Examples of such compounds are 2-naphtol orange; Allura Red AC; and Fast Yellow AB.

Even more preferably, the labeling compound is an azo compound having the formula

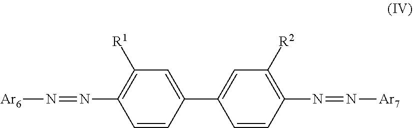

wherein Ar₆ and Ar₇ are the same or different (hetero)aryl groups; and R¹ and R² are each independently selected from hydrogen, methyl, ethyl, methoxy, amino, hydroxyl, sulfhydryl and sulfonate. Ar₆ and Ar₇ have the same definition as Ar₃ and Ar₄ described above.

Ar₆ and Ar₇ are preferably each individually selected from phenyl, biphenyl, naphtyl, benzothiazole and phenazine, wherein said phenyl, biphenyl, naphtyl, benzothiazole and phenazine may optionally be substituted with one or more groups selected from methyl, ethyl, phenyl, methoxy, nitro, amino, dimethylamino, diethylamino, hydroxyl, sulfonate, sulfhydryl and —N═N—Ar₅. The group —N═N—Ar₅ is as defined above. Examples of compounds of formula (IV) are Chicago Sky blue 6B and Diamine Green B.

In a preferred embodiment of the compound of formula (IV), Ar₆ and Ar₇ are each individually selected from phenyl and naphtyl, wherein said phenyl and napthyl may optionally be substituted with one or more groups selected from methyl, ethyl, methoxy, nitro, amino, hydroxyl, and sulfonate. More preferably, at least one of Ar₆ and Ar₇ is napthyl, which naphtyl is optionally substituted with one or more groups selected from methyl, ethyl, methoxy, amino, hydroxyl, and sulfonate. Examples of such compounds are 2-naphtol orange; Allura Red AC; and Fast Yellow AB. An example of such a compound is Chicago Sky Blue 6B.

The above compounds can be in neutral form or provided as salts thereof, e.g. sodium salts.

In case the visualizing agent is a dye or azo dye, it is preferably a vital dye. The term “vital dye” as used herein refers to a dye which has a sufficient coloring, or staining capacity at a concentration which is physiologically and toxicologically acceptable (e.g. without clinically significant interference with the cell metabolism). Hence, such a dye can be used in an (in-vivo) environment of living cells and tissues. In other words, the minimum amount of dye which is necessary to provide sufficient staining for a useful coloring to be visible should be low to such an extent that no, or hardly any, adverse toxic effects occur.

The invention is further directed to a complex of the visualizing agent and hyaluronan. As described above, the visualizing agent binds to the hyaluronan. A complex of these two may be used in diagnostics, therapeutics, surgery and cosmetics. In particular, such a complex may be used in the form of an ‘injectionable’, as described below.

In a second aspect, the invention is directed to a visualizing composition comprising a polyoxazoline according to formula (I) and one or more labeling compounds. The polyoxazoline and the labeling compounds are as described above.

The ratio between the molar amount of labeling compound and the molar amount of polyoxazoline present in the composition is preferably between 100/1 and 1/1, more preferably between 50/1 and 2/1, even more preferably between 30/1 and 10/1. Since multiple labeling compounds can bind to one polyoxazoline molecule, said molar amount should not be too low. Depending on the length of the polyoxazoline, a higher or lower amount of the labeling compound may be used.

The visualizing composition is preferably a liquid composition, more preferably an aqueous composition, even more preferably an aqueous solution. The inventors found that the labeling compound and the polyoxazoline may spontaneously form the visualizing agent according to the invention when dissolved in water. This is for example the case when the labeling compound is an azo compound. An azo compound will spontaneously bind to the polyoxazoline in water.

Polyoxazoline may be present in the liquid composition in an amount of 0.1-15 wt. %, preferably 0.5-10 wt. %, even more preferably 1.0-5.0 wt. %. High concentrations may not be desirable in view of the viscosity of the composition. High polyoxazoline concentrations may be difficult to handle and apply due to a high viscosity.

The labeling compound may be present in the liquid composition in an amount of 0.001-5 wt. %, preferably 0.001-1.0 wt. %, even more preferably 0.01-0.5 wt. %, even more preferably 0.02-0.2.

The visualizing agent according to the invention may be present in the liquid composition in an amount of 0.1-20 wt. %, preferably 0.5-10 wt. %, even more preferably 1.0-5.0 wt. %.

The viscosity of the liquid composition is preferably at least 2.0 mPa·s, more preferably at least 5 mPa·s, even more preferably 10 mPa·s. Furthermore, the viscosity of the staining composition is preferably less than 50 mPa·s, more preferably less than 20 mPa·s. Viscosity values can be determined using a rheometer at a temperature of 298 K. If necessary, the viscosity can be increased by including a viscosity enhancing compound, such as e.g. polyethylene glycol (PEG). In case of a viscoelastic composition, the viscosity will typically be higher.

The polyoxazoline in the visualizing composition is preferably not cross-linked, or at least not to any significant degree. Crosslinking may interfere with the binding to hyaluronan.

The visualizing composition may also be provided in solid form. To such a composition, a liquid, e.g. an aqueous solution can be added later to prepare a fresh liquid composition.

In the composition according to the invention, the azo compounds may be provided in their neutral form, or as a salt or hydrate of the compound, e.g. a sodium salt.

The visualizing composition may be an ophthalmic composition for visualizing the vitreous. In this case, the labeling compound is a dye compound.

The visualizing composition may be a dental composition for visualizing the gingiva. In this case, the labeling compound may be a dye compound or a contrast agent.

The visualizing composition may be a contrast medium for visualizing hyaluronan containing tissue. In this case, the labeling compound may be an isotope label compound (e.g. comprising ¹¹¹In) or an MRI contrast agent (e.g. comprising Gd).

The visualizing composition may be a cosmetic coloring agent for coloring synthetic hyaluronan tissue. In this case, the labeling compound may be a pigment suitable for coloring the skin.

In preferred embodiment, the visualizing composition according to the invention further comprises hyaluronan. Such a composition may be referred to herein as an ‘injectionable’, as the composition is typically administered to a subject by injection. A visualizing composition comprising hyaluronan can be used in applications wherein the subject is to be administered hyaluronan. Such injectables containing hyaluronan are known in the art and the skilled person will know how to prepare them. By including a visualizing agent according to the invention in such compositions, the administration of hyaluronan to the patient can be monitored. As described above, the polyoxazoline present in the visualizing agent will bind hyaluronan. Accordingly, a visualizing composition comprising hyaluronan will comprise a complex of the visualizing agent and hyaluronan. The amount of hyaluronan present in the composition may be considerably larger than the amount of visualizing agent. Accordingly, the composition may in such a case comprise both hyaluronan that is bound to the visualizing agent, and hyaluronan that is not bound to the visualizing agent. An injectionable may for example be used for surgical applications. An example of such an injectionable is a viscoelastic composition, which may be used in ocular surgery (e.g. cataract surgery, glaucoma surgery, corneal transplantation, vitreoretinal surgery), e.g. to stabilize the anterior chamber. An injectionable may also be used for therapeutic or diagnostic applications. For example, an injectionable of the invention may be used to administer hyaluronan to the hyaline cartilage within a joint. An injectionable may also be used for cosmetic applications. For example, a hyaluronan containing visualizing composition may comprise a dye as the labeling compound for coloring the skin. Such a composition can be injected into the skin.

The hyaluronan present in the visualizing composition of the invention may be of any suitable molecular weight. The molecular weight of hyaluronan is generally within the range of 50,000 to 8,000,000 g/mol, although there are reports of molecular weights as high as 13,000,000 depending on the source, method of isolation and method of determination.

In case the visualizing composition is a viscoelastic composition for eye surgery, the properties of the visualizing composition may be similar as described below for the ocular staining composition. Hyaluronan is a viscoelastic substance. As such, the skilled person will know how to prepare a viscoelastic visualizing composition. The zero shear viscosity of hyaluronan generally varies from 50,000 to 10,000,000 centipoise. Zero shear viscosity values can be determined using a rheometer at a temperature of 298 K.

In a particularly preferably embodiment, the visualizing composition is suitable for staining the vitreous body. Such a composition may be referred to herein as an ocular staining composition.

An ocular staining composition is preferably an aqueous composition, preferably an aqueous solution. The amount and ratio of the polyoxazoline and the labeling compound are as defined above for the general composition of the invention, unless specifically mentioned otherwise. The viscosity mentioned above for the liquid composition also applies to the ocular staining composition.

The labeling compound in an ocular staining composition is preferably an azo dye as defined above. The azo dye is preferably a vital dye. In particular, an azo dye according to formula (III) or (IV) as defined above is suitable for an ocular staining composition. These dyes are capable of visualizing the vitreous body by staining the periphery of the vitreous body, without staining the retina.

The staining composition may further comprise a salt. The ocular staining composition is preferably isotonic with ocular fluid. For this purpose, the ocular staining composition may comprise a salt to adjust its osmolarity to a suitable value. The staining composition of the invention preferably has an osmolarity between 250 and 400 mosmol/L, preferably 300-330 mosmol/L, for example 315 mosmol/L. The skilled person will be able to calculate the amount of salt needed to achieve this.

The salt may be chosen from the group consisting of sodium chloride, potassium chloride, calcium chloride, magnesium chloride, or a combination thereof. To provide the staining composition with a salt, the staining composition may comprise a salt solution. Suitable examples are Balanced salt solution or Hartmann's lactated Ringer's solution (see also Nuijts R M M A, Edelhauser H F, Holley G P, “Intraocular irrigating solutions: a comparison of Hartmann's lactated Ringer's solution, BSS and BSS plus”, Clin. Exp. Ophtamol., vol. 233 (1995), pp. 655-661).

It is further preferred that the liquid staining composition has a neutral or slightly basic pH, i.e. a pH of 6.5-8. Preferably, the composition has a pH of 7.2-7.7. To maintain a stable pH, the staining composition may comprise a buffer, preferably a salt buffer, which is suitable for ophthalmic applications. An example of a suitable buffer is phosphate buffered NaCl.

The concentration of the dye in the ocular staining composition is preferably 0.001-2 wt. %, more preferably 0.01-1 wt. %, even more preferably 0.05-0.5 wt. %, even more preferably 0.1-0.5 wt. %, based on the total weight of the staining composition. Within this range, the concentration may be adapted to the toxicity and coloring characteristics of the dye used. It is preferred that such an amount is chosen that an optimal staining effect is achieved, while at the same time the risk of possible damage to the eye or any part thereof due to the toxicity of the dye is minimized.

The ocular staining composition may comprise a second dye compound, which compound is not bound to polyoxazoline. The second dye is preferably a dye other than an azo dye. Preferably, the second dye is not capable of binding to polyoxazoline. Such a second dye can be used to stain an ocular tissue other than the vitreous body. The second dye may be selected from the group consisting of brilliant blue G, methylene blue, patent blue V, indocyanine green, crystal violet, safranin, fluorescein and rose bengal.

Each component in the ocular staining composition preferably has a concentration in the staining composition that is physiologically and toxicologically acceptable. In other words, the minimum amount of each component in the staining composition should be sufficiently low such that no, or hardly any, adverse toxic effects occur. Preferably, each component in the staining composition is not, or at least hardly, toxic for the retina and adjacent structures. It is further preferred, that the content of each component in the staining composition present in the eye, shortly after the eye surgery poses hardly any risk of the patient experiencing any side-effects from the use of the staining composition.

The ocular staining composition may be used in the treatment of staining an ocular tissue or part of an ocular tissue, in particular the vitreous body. The staining treatment may be part of eye surgery. As also noted above, staining of at least part of an ocular tissue may be used in eye surgery to facilitate the work of the surgeon by making it easier for him to visually distinguish one ocular tissue from the other. The staining composition is typically applied to the surface of the ocular tissue to be stained. The staining composition may then be allowed to spread over and/or through this tissue by allowing the staining composition to sink onto or penetrate the tissue, e.g. under the force of gravity. In case of staining the vitreous body, the ocular staining composition in particular stains the outer layer and/or outer surface or of the vitreous body. This can be achieved without staining the retina. This application is further discussed below with respect to the fifth and sixth aspect of the invention.

In a third aspect, the invention is directed to a visualizing composition comprising the visualizing agent according to the invention. Also provided is a visualizing composition comprising a visualizing agent comprising one or more labeling compounds that are molecularly bound to a polyoxazoline molecule and hyaluronan bound to the polyoxazoline molecule, the polyoxazoline compound having a repeat unit according to the formula

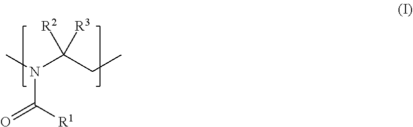

as defined herein. In one embodiment, the visualizing composition further comprises hyaluronan that is not bound to the visualizing agent. The composition may further be similar in composition as the visualizing composition of the second aspect.

In a fourth aspect, the invention is directed to a method for preparing the visualizing agent. The method comprises the step of providing a polyoxazoline of formula (I) and a labeling compound; and mixing the polyoxazoline and the labeling compound in water to form an aqueous mixture. The labeling compound may be provided in its neutral form, or as a salt or hydrate thereof. In case the labeling compound is an azo compound according to any one of formulas (II), (III) and (IV), the visualizing agent according to the invention will form upon mixing. Particular good results have been obtained using an azo compound according to formula (III), which resulted in very stable molecular complex.

Mixing may be conducted at a temperature of 0-100° C., preferably 1-50° C., even more preferably 5-30° C., for example at room temperature.

The time for the bond between the azo dye and the polyoxazoline to establish was less than 3 ins, as determined using stopped flow kinetics. Nevertheless, mixing should preferably be conducted for a sufficiently long time for the azo compound to be able to dissolve.

The polyoxazoline is preferably provided in the form of an aqueous solution. The azo compound may also be provided in the form of an aqueous solution.

Since multiple labeling compounds can bind to one polyoxazoline molecule, an excess of labeling compound should be used. Preferably, the molar amount of labeling compound used in the method may be 1-100 times, preferably 2-50 times, even more preferably 10-30 times the molar amount of polyoxazoline.

The resulting aqueous mixture preferably has a concentration of polyoxazoline and labeling compound as defined above for the visualizing composition.

The resulting aqueous mixture may have a pH of 5 to 9, preferably 6.5 to 8, more preferably 7.2 to 7.8. This pH allows for the formation of stable molecular complex when an azo compound according to formula (II), (III) or (IV) is used.

The method described above can also be suitably used for preparing the visualizing composition according to the invention, and in particular for preparing the ocular staining composition.

The method may further comprise the step of adding additional components in the water before, during or after mixing. For example, a salt or salt buffer may be added in this way. The method may also comprise the step of adding a second dye compound to the aqueous mixture.

Alternatively, the visualizing agent according to the invention may be prepared in a method wherein a labeling compound is covalently attached to a polyoxazoline. The visualizing agent may also be prepared in a method wherein a labeling compound is covalently attached to an oxazoline monomer and wherein the resulting visualizing monomer is subsequently used in a polymerization reaction to obtain the visualizing agent.

In a fifth aspect, the invention is directed to the use of the visualizing agent or the visualizing composition according to the invention for visualizing hyaluronan. The hyaluronan may be synthetic hyaluronan or hyaluronan tissue. The use may include subjecting the visualizing agent or visualizing composition with ultraviolet, MRI, X-radiation (Röntgen radiation), NMR, PET or infrared. The use preferably comprises administering the visualizing agent to the eye and/or the vitreous body. Also provided is a method for visualizing hyaluronan, comprising administering the visualizing agent according to the invention to the eye and/or the vitreous body. For example, the invention provides for a use of or a method for visualizing a hyaluronan tissue or part thereof, comprising the steps of bringing the visualizing composition according to the invention or the visualizing agent according to the invention in contact with the hyaluronan tissue. The use or method may further comprise activating the visualizing agent, e.g. by subjecting the contacted tissue with ultraviolet, MRI, X-radiation (Röntgen radiation), NMR, PET or infrared.

The visualizing agent may be brought in contact with the hyaluronan tissue by applying the visualizing composition of the invention to the surface of the hyaluronan tissue. The visualizing agent may also be injected onto or into the hyaluronan tissue. In case of an ocular staining composition, the method for visualizing the hyaluronan tissue or part thereof (i.e. the vitreous body) comprises the steps of applying the staining composition of the invention to the surface of the vitreous body.

In a sixth aspect, the invention is directed to the use of the visualizing agent or the visualizing composition according to the invention in diagnostics, therapeutics, surgery and cosmetics. The visualizing agent or the visualizing composition may be used in these applications to visualize hyaluronan, as described above for the fifth aspect. Examples of such uses of the visualizing agent or the visualizing composition are described in further detail below.

One particular preferred use of the visualizing agent or visualizing composition is in eye surgery, wherein said agent or composition can be used to stain the vitreous body or part thereof. In this respect, the invention is in particular directed to the ocular staining composition according to the invention for use in a method of eye surgery comprising staining the vitreous body or part thereof, and performing surgery on the stained vitreous body or its surrounding tissue. The details of the ocular staining composition and the visualizing agent present therein are as described above. The ocular staining composition may be used to stain the vitreous body so as to distinguish it from a surrounding ocular tissue during surgery. In particular, the outer surface or periphery of the vitreous body is stained. The staining does typically not affect or stain the ocular tissues surrounding the vitreous body. Staining can be achieved by contacting the vitreous body with the visualizing agent or ocular staining composition, e.g. as described above for the fifth aspect of the invention.

The eye surgery typically comprises the removal of at least part (but preferably all) of the vitreous body or at least part of a tissue surrounding the vitreous body. Tissues surrounding the vitreous body are for example the retina and retinal membranes.

Surgical intervention for vitrectomy usually aims to completely remove all remnants of the vitreous body. This typically includes removing the scaffold that is believed to facilitate cells involved in scar formation (fibrosis). Although various techniques and instruments may be used for ‘removal of vitreous strands’, the common problem with all of these approaches is that the vitreous body or parts thereof can not be clearly identified by the surgeon. Vitreous removal is therefore currently performed by judging the tissue response of various surrounding anatomical structures (no tissue movement upon provocation), by negative staining with vital dyes that stain anatomical tissue structures (in the absence of staining, remnant vitreous may be present). The present invention solves the problem by providing a way of staining the vitreous body.

The surgery in which the ocular staining composition is used may be vitreo-retinal surgery. Such surgery commonly comprises vitrectomy or retinal surgery or both. In vitrectomy, at least part of the vitreous body is manipulated and/or removed. In retinal surgery, at least part of the retina or a retinal membrane is manipulated and/or removed. Examples of retinal membranes are the epiretinal membrane and the inner limiting membrane.

In vitrectomy, the tissue on which surgery is performed is the vitreous body. At least part or all of the vitreous body is manipulated and/or removed in vitrectomy. The most common example is pars plana vitrectomy. This type of vitrectomy can be conducted with or without manipulation and/or removal of surrounding ocular structures (for example retinal membrane removal). Another example of vitrectomy is anterior vitrectomy. Anterior vitrectomy comprises removing small portions of the vitreous body from the anterior segment of the eye.

Hence, in one preferred embodiment, the eye surgery comprises or is capsulorhexis.

The staining composition may be removed from the eye together with the vitreous body.

Conditions that can be treated with vitreo-retinal surgery, an in particular with vitrectomy, comprise retinal detachment, macular pucker, diabetic retinopathy, macular holes, vitreous hemorrhage and vitreous floaters.

The staining step may be conducted by applying the staining composition of the invention to the outer surface of the vitreous body. This can be done by using a cannula or syringe, preferably a blunt cannula. The cannula or syringe can be placed upon the outer surface of the lens capsule to apply the staining composition.

The amount of staining solution used to stain the vitreous body may be in the range of 0.01 to 1.0 mL, preferably in the range of 0.1 to 0.3 mL. Repeated application of the staining solution may be used during the same surgical session.

Another preferred use of the visualizing agent or visualizing composition in eye surgery is its application as a viscoelastic composition. It is known to inject a viscoelastic compositions in the anterior chamber in order to stabilize said chamber during eye surgery. The viscoelastic composition acts as a spacer maintainer, i.e. the composition is for maintaining a sufficiently large space in the anterior chamber during surgery. In this way, eye surgery can be facilitated. An example is the performance of a capsulorhexis. The injection of a viscoelastic composition in the anterior chamber facilitates the creation of a circular opening in the crystalline lens capsule during cataract surgery. Furthermore, a viscoelastic composition can be injected in the anterior chamber of the eye to improve contrast between the lens capsule and the surrounding tissue. Such applications are e.g. described in EP 1 132 065. The procedures described above can also be conducted with the ocular staining composition of the invention. In this case, the composition comprises hyaluronan and is in the form of a viscoelastic composition. Accordingly, the invention provides a viscoelastic ocular staining composition for use in a method of eye surgery comprising injecting the viscoelastic ocular staining composition in the anterior chamber of the eye; and performing surgery on the ocular structures surrounding the injected viscoelastic ocular staining composition. The visualizing agent in this application preferably only stains the viscoelastic composition, while not staining the ocular tissue. The surgery may be cataract surgery, glaucoma surgery, corneal transplantation or vitreoretinal surgery. In particular, cataract surgery may be facilitated by using a viscoelastic ocular staining composition. In this type of surgery, the lens capsule is manipulated by the surgeon during surgery. At the end of the surgery, the viscoelastic liquid can be removed by rinsing, e.g. with salt solution.

A viscoelastic ocular staining composition according to the invention comprises hyaluronan. Hyaluronan provides the composition with viscoelastic properties. The viscoelastic ocular staining composition may have similar properties as described above for the ocular staining composition. The viscoelastic ocular staining composition may comprise a second dye. Such a dye may stain ocular tissue. This is advantageous as the visualizing agent in this application is only for staining the viscoelastic ocular staining composition. Further examples of using the visualizing agent or the visualizing composition according to the invention in diagnostics, therapeutics, surgery and cosmetics are described below.

The visualizing agent or the visualizing composition may be for use in surgery, e.g. ocular surgery (as described above) or dental surgery. For example, the invention provides for a visualizing agent according to the invention or a visualizing composition according to the invention for use in dental surgery comprising staining the gingiva (gums) with said visualizing agent or composition. For example, a dye may be used for staining to improve visual for the surgeon. Visualization may also be achieved by using a labeling compound that is detectable by X-ray.

The term “performing surgery” as used herein may refer of the step in surgery of manipulating and/or removing tissue, which step is typically conducted by the surgeon. The tissue may refer to ocular tissue or ocular structures (such as an ocular membrane or the vitreous body) or the gingiva.

The visualizing agent or visualizing composition can be used in diagnostics. In this case, the labeling compound may be an isotope label compound (e.g. comprising ¹¹¹In) or a PET or MRI contrast agent (e.g. comprising Gd). Examples of diagnostic applications are diagnosing the gingiva associated conditions or diseases (e.g. gingivitis) or hyaline cartilage associated conditions or diseases in the joints.

The visualizing agent or visualizing composition can be used in cosmetics. The labeling compound may be a pigment suitable for coloring the skin. The visualizing agent or visualizing composition can for example be administered by injection in the skin.

The visualizing agent or visualizing composition can be used in therapeutics, e.g. in pharmaceutics. In particular, the use of ‘injectionables’, wherein the visualizing agent is bound to hyaluronan, is a preferred application. Such compositions can be used in treatments wherein hyaluronan is to be administered (e.g. conditions or diseases associated with hyaluronan deficit, e.g. associated with hyaline cartilage). The visualizing agent provides for the possibility of monitoring the treatment during or after administration.

The invention is further directed to a method of conducting X-ray, infrared, PET or MRI measurements on a subject, wherein the subject has hyaluronan containing tissue that has been bound with the visualizing agent according to the invention or with the visualizing composition according to the invention. The tissue may be cartilage, articular cartilage or hyaline cartilage and may be located in the joint of the subject. The tissue may also be the gingiva.

The invention is further directed to the use of the polyoxazoline as defined above for binding an azo dye and subsequent removal of the dye from stained tissue. Accordingly, the invention is directed to a polyoxazoline having a repeat unit according to formula (I) as defined above for use in surgery, wherein the surgery comprises staining tissue with an azo dye, performing surgery, contacting the azo dye with said polyoxazoline; and removing the resulting complex formed between the azo dye and said polyoxazoline. The surgery may be eye surgery, wherein ocular tissue is typically stained using one or more dyes. The ocular tissue may e.g. be an epiretinal membrane, an inner limiting membrane or the lens capsule. It was found that polyoxazoline binds to these dyes more strongly than it does to the tissue, thereby effectively removing the dye from the tissue. The removal is typically conducted during surgery after the step of manipulating and/or removing ocular tissue.

The invention is further directed to a drug delivery complex. The drug delivery complex is the same complex as the visualizing agent described above, except that instead of a labeling compound, an active ingredient is bound to the polyoxazoline molecule. The active ingredient may comprise an azo moiety according to formula (II) as described above.

The invention is further directed to a kit of parts comprising one or more labeling compounds and a polyoxazoline molecule having a repeat unit according to formula (I).

With respect to the above applications, the body is generally capable of removing the visualizing agent from the body via the kidneys.

Although the invention has been described above with respect to hyaluronan and hyaluronan containing tissue, it is expected that the visualizing agent is also capable of visualizing mucopolysaccharides in general. Mucopolysaccharides (also called glycosaminoglycan), are polysaccharides consisting of a repeating disaccharide unit. The repeating unit typically consists of an amino sugar (N-acetylglucosamine or N-acetylgalactosamine), a uronic sugar (glucuronic acid or iduronic acid) or galactose. Examples of mucopolysaccharides are hyaluronan (also called hyaluronic acid), heparin sulfate, heparan sulfate, chondroitin sulfate, dermatan sulfate and keratin sulfate. In view of the similarity in structure with other mucopolysaccharides, it is expected that the visualizing agent according to the invention can also be used to visualize mucopolysaccharides containing tissue. The different aspects described above with respect to visualizing hyaluronan may therefore also apply to visualizing the other mucopolysaccharides described above.

Features may be described herein as part of the same or separate aspects or embodiments of the present invention for the purpose of clarity and a concise description. It will be appreciated by the skilled person that the scope of the invention may include embodiments having combinations of all or some of the features described herein as part of the same or separate embodiments.

The invention will be explained in more detail in the following, non-limiting examples.

### EXAMPLE 1: BINDING OF POLYOXAZOLINE WITH AZO COMPOUNDS

Absorbance tests were conducted to establish the bond between polyoxazoline and chicago sky blue (an azo compound).

The first sample was prepared by dissolving Chicago Sky Blue (CSB) in a phosphate buffered saline (PBS).

The second sample was prepared by dissolving CSB in a phosphate buffered saline comprising 5% poly(2-ethyl-2-oxazoline) with a Mw of 5,000 g/mol (PEtOx).

The results are shown in FIG. 7. The first sample (CSB in PBS) had a maximum Absorbance (Aₘₐₓ) at a wavelength (λₘₐₓ) of about 610 nm, while the second sample (CSB in PBS+PEtOx) had an Aₘₐₓ at about 640 nm. The shift in Aₘₐₓ can be attributed to the binding of CSB with PEtOx.

A third and fourth sample were prepared having the same composition as the first and second sample respectively, except that NaCl was added such that the sample contained 1M NaCl. The shape and (λₘₐₓ) of samples 1 and 3 were the same. The shape and (λₘₐₓ) of samples 2 and 4 were also the same. From this experiment, it could be concluded that the presence of NaCl does not have much effect on the bond between PEtOx and CSB. The bond is therefore unlikely to be ionic.

A fifth sample was prepared having the same composition as the second sample, except that poly(2-methyl-2-oxazoline) was used in stead of poly(2-ethyl-2-oxazoline). The results are shown in FIG. 8. It can be concluded that a similar bonding is established when using these two types of polyoxazoline.

### EXAMPLE 2: STAINING OF THE VITREOUS BODY

Ocular staining compositions were prepared by dissolving an azo compound (100.mg) in PBS (100 ml) containing 1-10% poly(2-ethyl-2-oxazoline) with a Mw of 5,000 g/mol. These staining compositions were subsequently used to stain a vitreous body from a surgically removed human eye. The composition was applied by brining it in contact with the outer surface of the vitreous body.

The following azo compounds were used in the test:
allura red (FIG. 9a)
2-naphtol orange (FIG. 9b)
thiazole yellow (FIG. 9c)
Janus Green (FIG. 9d)
Chicago Sky Blue (FIG. 9e).

Photographs were taken of the result of staining the vitreous body with the azo dyes. The results are shown in FIG. 9. The references to the azo dye used in the Figure are indicated above.

It was concluded that all tested azo dyes were capable of staining the vitreous body when present in an aqueous solution with 2-ethyl-2-oxazoline.

### EXAMPLE 3: EFFECT OF POLYOXAZOLINE MW AND NACL ON STAINING

Ocular staining solutions comprising CSB and 2-ethyl-2-oxazoline (PEtOx) with different Mw were prepared. The molecular weight of the PEtOx was 50,000 g/mol (sample a), 25,000 g/mol (sample b) and 5,000 g/mol (sample d). Further, an ocular staining solution was prepared similar to sample d, but with the addition of NaCl to a concentration of 1M (sample c).

Photographs were taken of the staining results, which are shown in FIG. 10. The petri dishes in FIG. 10 contain the results of the vitreous body stained with sample a (left), sample b (middle left), sample c (middle right) and sample d (right).

It was concluded that all ocular staining solutions were capable of visualizing the vitreous body. The intensity of staining was found to be highest for Mw 50,000 (sample a). The presence of NaCl did not seem to affect the staining capability of the solution much.

1. A visualizing agent for visualizing hyaluronan comprising one or more labeling compounds that are molecularly bound to a polyoxazoline molecule having a repeat unit according to the formula 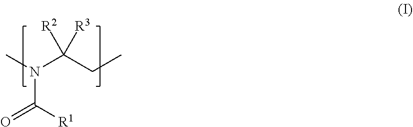
wherein R¹ is an alkyl group or a phenyl group, wherein said alkyl or phenyl is optionally substituted with one or more substituents selected from the group consisting of halogen, amino (—NH₂), nitro (—NO₂), carboxyl (—COOH), alkoxy (—OR), sulfonate (—SO₃⁻), hydroxyl (—OH) and sulfhydryl (—SH); and wherein said phenyl may additionally or alternatively also be optionally substituted with one or more alkyl groups; and
wherein R² and R³ are each individually selected from the group consisting of hydrogen, alkyl, halogen, amino (—NH₂), nitro (—NO₂), carboxyl (—COOH), alkoxy (—OR), sulfonate (—SO₃⁻), hydroxyl (—OH), imino (═NH) and sulfhydryl (—SH).

2. Visualizing agent according to claim 1, wherein the labeling compound is an azo compound, preferably having an azo moiety according to the formula [symbol]
Ar₁—N═N—Ar₂ [symbol] (II),
wherein Ar₁ and Ar₂ are the same or different aromatic rings.

3. Visualizing agent according to claim 2, wherein the labeling compound is an azo compound according to formula (III)
Ar₃—N═N—Ar₄ (III)
wherein Ar₃ and Ar₄ are the same or different (hetero)aryl groups; wherein Ar₃ and Ar₄ are each individually selected from phenyl, biphenyl, naphtyl and phenazine, wherein said phenyl, biphenyl, naphtyl and phenazine may optionally be substituted with one or more groups selected from methyl, ethyl, phenyl, methoxy, nitro, amino, dimethylamino, diethylamino, hydroxyl, sulfonate, sulfhydryl and —N═N—Ar₅, wherein the group —N═N—Ar₅ is an azo group, wherein Ar₅ is selected from phenyl and naphtyl, wherein said phenyl and naphtyl may optionally be substituted with one or more groups selected from methyl, ethyl, methoxy, nitro, amino, hydroxyl and sulfonate and sulfhydryl;
or an azo compound according to formula (IV) 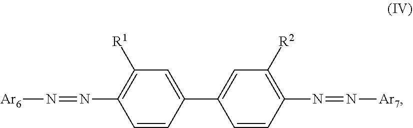
wherein Ar₆ and Ar₇ are the same or different (hetero)aryl groups; and R¹ and R² are each independently selected from hydrogen, methyl, ethyl, methoxy, amino, hydroxyl, sulfhydryl and sulfonate; and wherein Ar₆ and Ar₇ are preferably each individually selected from phenyl, biphenyl, naphtyl and phenazine, wherein said phenyl, biphenyl, naphtyl and phenazine may optionally be substituted with one or more groups selected from methyl, ethyl, phenyl, methoxy, nitro, amino, dimethylamino, diethylamino, hydroxyl, sulfonate, sulfhydryl and —N═N—Ar₅, wherein the group —N═N—Ar₅ is as defined above.

4. Visualizing agent according to claim 1, wherein the labeling compound is selected from the group consisting of a vital dye, a radiocontrast agent, an MRI contrast agent, a fluorescent compound, an isotope labeled compound, and a cosmetic coloring agent.

5. Visualizing agent according to claim 1, wherein the labeling compound is selected from Trypan Blue, Chicago Sky Blue 6B, Janus Green B, Allura Red, Fast Yellow AB, 2-naphtol orange, diamine green, coomassie blue, naphtol blue black, tartrazine, scarlet red, thiazole yellow G, and sudan black B.

6. Visualizing agent according to claim 1, wherein the polyoxazoline is selected from poly(2-ethyl-2-oxazoline) and poly(2-methyl-2-oxazoline).

7. Visualizing agent according to claim 1, wherein the polyxoazoline has a molecular weight of 200-500,000 g/mol. preferably 500-100,000 g/mol.

8. Visualizing agent according to claim 1, wherein the agent comprises at least 5 compounds per polyoxazoline molecule, wherein each labeling compound is non-covalently bound to polyoxazoline.

9. A visualizing composition comprising the visualizing agent according to claim 1.

10. Visualizing composition according to claim 9, wherein the composition is a an ophthalmic composition for staining the vitreous, a dental composition for visualizing the gums, a contrast medium for visualizing hyaluronan containing tissue in the joints, or a cosmetic composition.

11. Visualizing composition according to claim 9, wherein the composition is an aqueous solution for staining the vitreous, which composition has a pH of 6.5-8 and a concentration of the visualizing agent of 0.5-10 wt. %.

12. Visualizing composition according to claim 9, wherein the composition further comprises hyaluronan.

13.-19. (canceled)

20. A method for visualizing hyaluronan, comprising administering the visualizing agent according to claim 1 to hyaluronan containing tissue.

21. The method according to claim 20, wherein the visualizing agent is administered to the eye and/or the vitreous body, the gingiva or hyaline cartilage.

22. The method according to claim 20 comprising eye surgery.

23. A method for eye surgery comprising administering a visualizing agent according to claim 1 to the eye of a subject.

24. The method according to claim 22 comprising staining the vitreous body of the eye or part thereof with the visualizing agent and performing surgery on the stained vitreous body or its surrounding tissue.

25. The method according to claim 23 comprising injecting a viscoelastic ocular staining composition comprising the visualizing agent and hyaluronan in the anterior chamber of the eye and performing surgery on the ocular structures surrounding the injected viscoelastic ocular staining composition.

26. The method according to claim 23 comprising administering a viscoelastic composition comprising a visualizing agent according to claim 1 and hyaluronan for stabilizing the anterior chamber during said eye surgery.

27. The method according to claim 20 comprising dental surgery comprising staining the gingiva (gums) with said visualizing agent.

28. A method of surgery comprising staining tissue with an azo dye, performing surgery, contacting the azo dye with a polyoxazoline having a repeat unit according to formula (I) as defined in claim 1, and removing the resulting complex formed between the azo dye and said polyoxazoline.
